(19) United States
(12) Patent Application Publication
(10) Pub. No.: US 2008/0108655 A1
(43) Pub. Date: May 8, 2008

(54) Drug composition and method for treating malaria and malignancy

(76) Inventor: Martin J. Smilkstein, Portland, OR (US)

Correspondence Address: KOLISCH HARTWELL, P.C., 520 SW YAMHILL STREET, Suite 200, PORTLAND, OR, 97204, US

(21) Appl. No.: 11/352,388

(22) Filed: Feb. 10, 2006

Related U.S. Application Data
(60) Provisional application No. 60/652,021, filed on Feb. 10, 2005.

(51) Int. Cl. A61K 31/135; A61K 31/47; A61P 33/06; A61P 35/00

(52) U.S. Cl. 514/313; 514/648

(57) ABSTRACT

A method of treating a malarial infection includes using orphenadrine as part of treating the infection. The method may use orphenadrine alone or in combination with another antimalarial drug, such as chloroquine, or with plural antimalarial drugs. Use of orphenadrine in this way may also be used in a method of providing prophylaxis against a malarial infection, and as part of a method of treating a malignancy.

### CROSS-REFERENCE TO RELATED APPLICATIONS

This application claims priority to U.S. Provisional Patent Application Ser. No. 60/652,021, which was filed Feb. 10, 2005 and entitled “Drug Composition and Method for Treating Malaria and Malignancy”, which is incorporated herein by reference.

### BACKGROUND

Since World War II, the drug chloroquine has been the most important treatment for the disease malaria. Unfortunately, the parasite Plasmodium which causes malaria, including the species most lethal to humans, Plasmodium faliciparum (P. faliciparum), has become progressively resistant to chloroquine. As a result, chloroquine treatment failures and in vitro chloroquine resistance have been documented throughout the majority of the world where malaria is found. Similarly, resistance to other antimalarial drugs including quinine, mefloquine, halofantrine, pyrimethamine-sulfadoxine, amodiaquine, atovaquone and others has also been noted.

For decades, since the first chloroquine-resistant malaria was documented, extensive research has been done to both understand the mechanism(s) by which drug resistance develops and to find new ways to effectively treat drug-resistant malaria. In addition to seeking new drugs to kill the parasite, efforts have been made to reverse chloroquine resistance and render resistant parasites sensitive to chloroquine.

The observation that multi-drug resistance in cancer cells could be reversed by the addition of other drugs led previous investigators to study whether an analogous result could be produced in drug-resistant malaria. The finding that verapamil partially restored chloroquine sensitivity to resistant P. falciparum led to subsequent discovery of a long list of diverse compounds, in several structural classes, with similar results. Several experimental drugs and many commonly used pharmaceutical products have shown antimalarial drug resistance-reversal (often termed chemosensitization) properties.

Some, like verapamil, are not candidates for clinical use for this purpose because of their known toxicity at concentrations needed to produce the desired effect. The tricyclic antidepressant, desipramine, reversed chloroquine-resistance in vitro and in a primate animal trial, but was ineffective in a human clinical trial. Several antihistamine drugs are effective in vitro and in rodent studies, and a few have reached clinical trials. The most promising has been chlorpheniramine (CP). In addition to its demonstrated effectiveness in vitro and in rodent malaria models, chlorpheniramine-chloroquine combination therapy has resulted in improved parasitological and clinical cure rates compared with chloroquine alone, and has been comparable to other combination drug protocols.

Despite the success of chlorpheniramine-chloroquine combination therapy, several shortcomings are evident. Chlorpheniramine, like most older antihistamines, consistently causes drowsiness which interferes with outpatient compliance and limits the dose which can be used. Secondly, a 3-day course of chlorpheniramine-chloroquine was inadequate. Either 7 days of chlorpheniramine, or the addition of a third drug was needed to safely treat resistant infections; either modification would complicate the regimen and increase the cost. Lastly, although the combination was far superior to chloroquine alone, there were small numbers of treatment failures in these studies. Taken in total, the evidence indicates that combination therapy with chlorpheniramine and chloroquine is close, but not close enough to being an effective weapon against chloroquine-resistant malaria.

Because structurally or functionally related drugs have also been shown to chemosenstitize drug-resistant malaria, the inventor has investigated other compounds seeking alternatives superior to chlorpheniramine

The example of chloroquine illustrates several reasons to again elevate the study of established antimalarial resistance-reversal from a mechanistic probe and research tool to serious clinical consideration. Unlike new drugs, chloroquine is widely and immediately available, inexpensive and its dosing and safety profile is extensively understood. As a result, for most of the malaria-affected world, restoration of chloroquine effectiveness would be the single most powerful possible antimalarial drug advance. Resistance “reversal” is possible because, unlike resistance to other drugs (e.g., pyrimethamine-sulfadoxine) from mutations which irreversibly prevent drug action, the mechanism of action of chloroquine persists even in resistant parasites. If enough chloroquine gains and retains access to its site of action, it is still effective, even in chloroquine-resistant parasites. This fact, clearly demonstrated in vitro, in vivo and in clinical trials leaves no doubt that the concept is sound; the challenge is to accomplish chloroquine resistance reversal in a manner that is effective, safe, inexpensive and feasible.

### SUMMARY OF THE INVENTIONS

Use of the drug orphenadrine, alone or in combination with other antimalarial drugs (e.g. chloroquine), to treat a malarial infection(s), provide prophylaxis against a malarial infection(s), and to treat a malignancy. These are new uses of orphenadrine, as are any combined formulations with existing or novel antimalarials or antimalignancy drugs.

Orphenadrine will be superior to chlorpheniramine, surpass a threshold of adequate clinical effect, and be several-fold more potent than chlorpheniramine. This conclusion is based on four factors:
in vitro, orphenadrine is approximately twice as potent as chlorpheniramine in chemosensitizing chloroquine-resistant P. falciparum strains W2 and Dd2 to chloroquine. In addition, the inventor has found that orphenadrine also increases sensitivity to quinine or mefloquine in strains resistant to those drugs;
orphenadrine protein binding is only approximately 20%, so that free (active) concentrations of orphenadrine are much higher than for chlorpheniramine at equal total concentrations; and
in previous human use, safely achievable concentrations of orphenadrine in clinical use are far higher than those of chlorpheniramine.
orphenadrine does not typically cause sedation

### BRIEF DESCRIPTION OF THE FIGURES

FIG. 1 depicts a pair of graphs showing the intrinsic antimalarial activity of orphenadrine, with the left graph showing orphenadrine being used on multi-drug resistant parasites (e.g. Dd2), and the right graph showing orphenadrine being used on non-resistant parasites (e.g. D6).

FIGS. 2-4 depict pairs of graphs, with the left graph of each pair showing the concentration of the antimalarial on the X axis, and the antimalarial EC50 values on the Y axis, and the right graph of each pair showing a representative isobologram from each of the experiments with lines predictive of EC_{50, 75 and 90} assuming the combinations were additive.

FIGS. 5-7 depict pairs of graphs similar to those for FIGS. 2-4, only each pair showing orphenadrine's effectiveness against a parasite resistant to quinine (FIG. 5; Dd2), and parasites resistant to mefloquine (FIGS. 6 -7; D6 and Dd2, respectively).

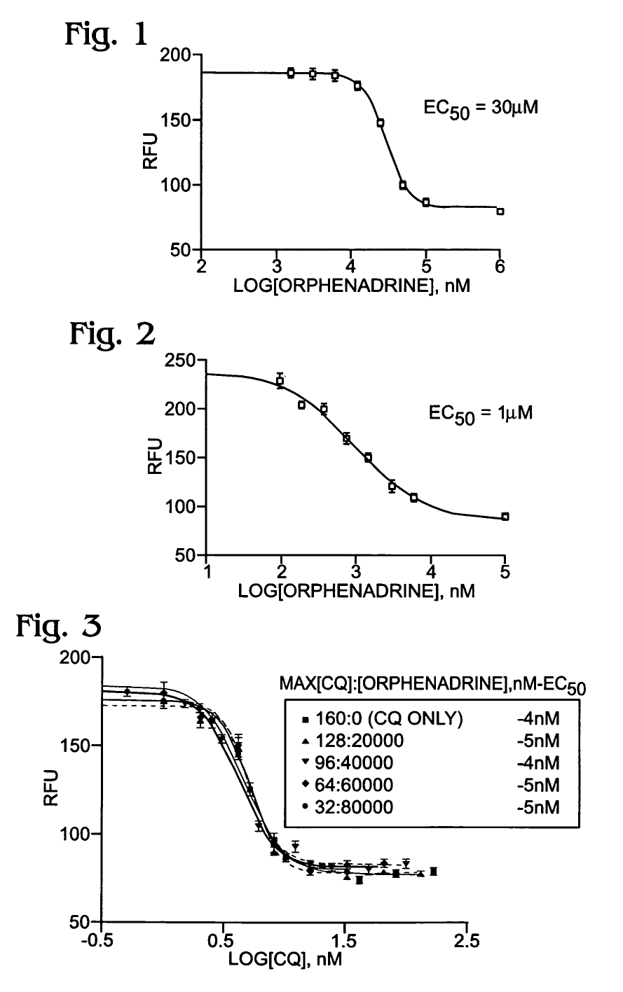

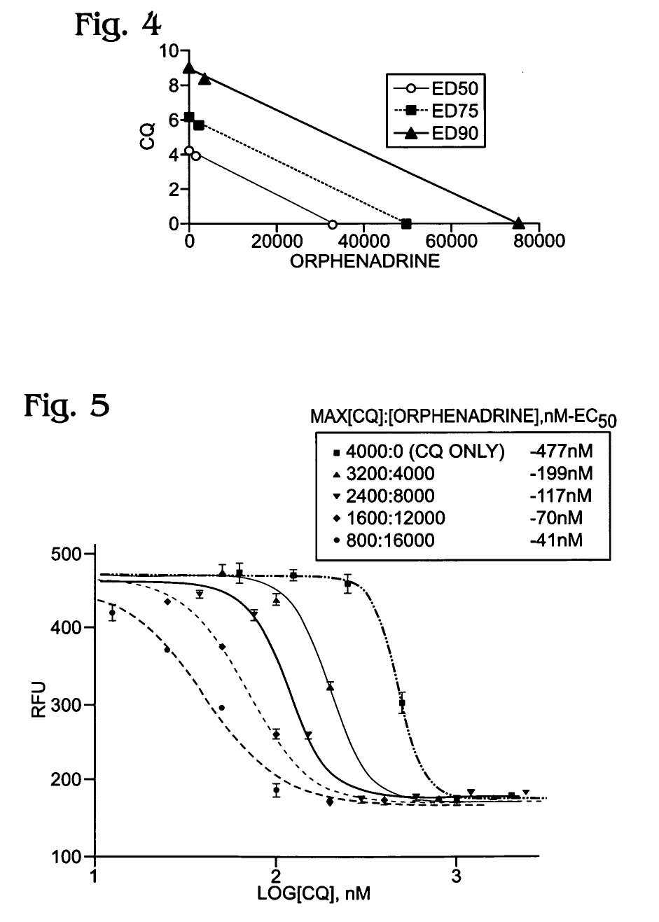

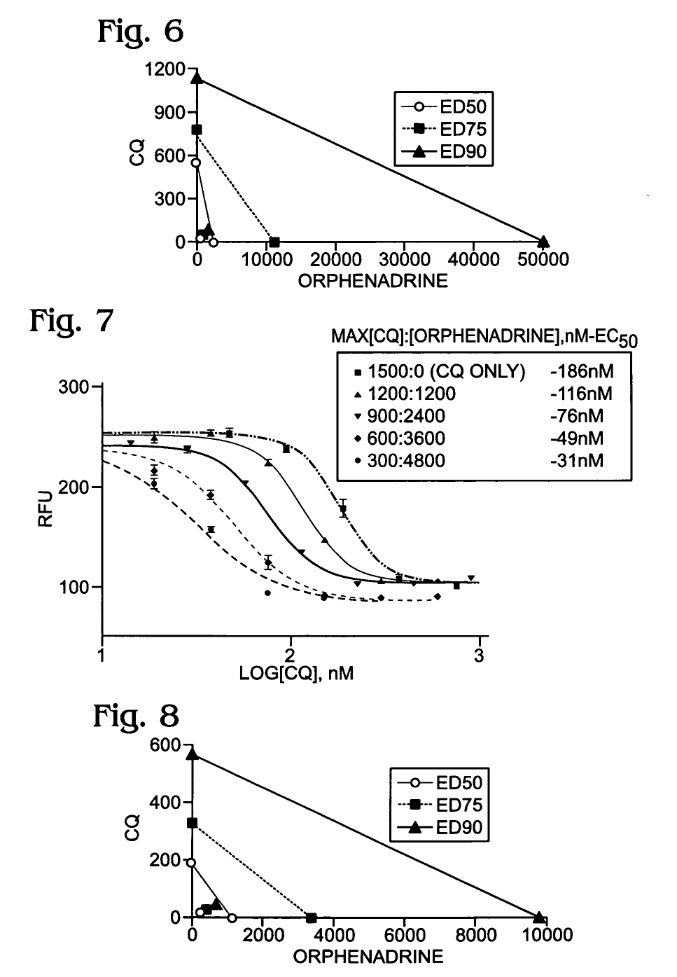

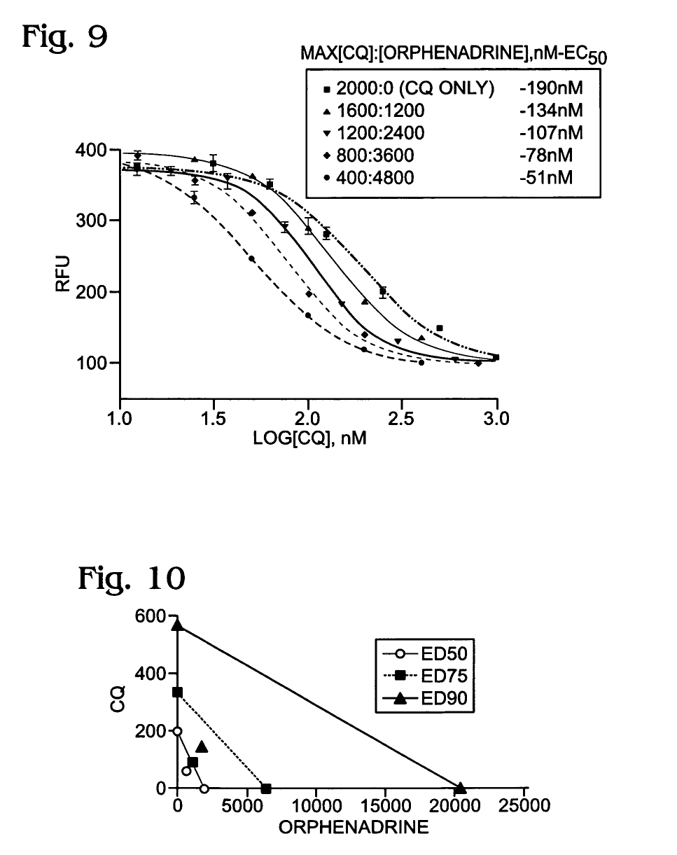

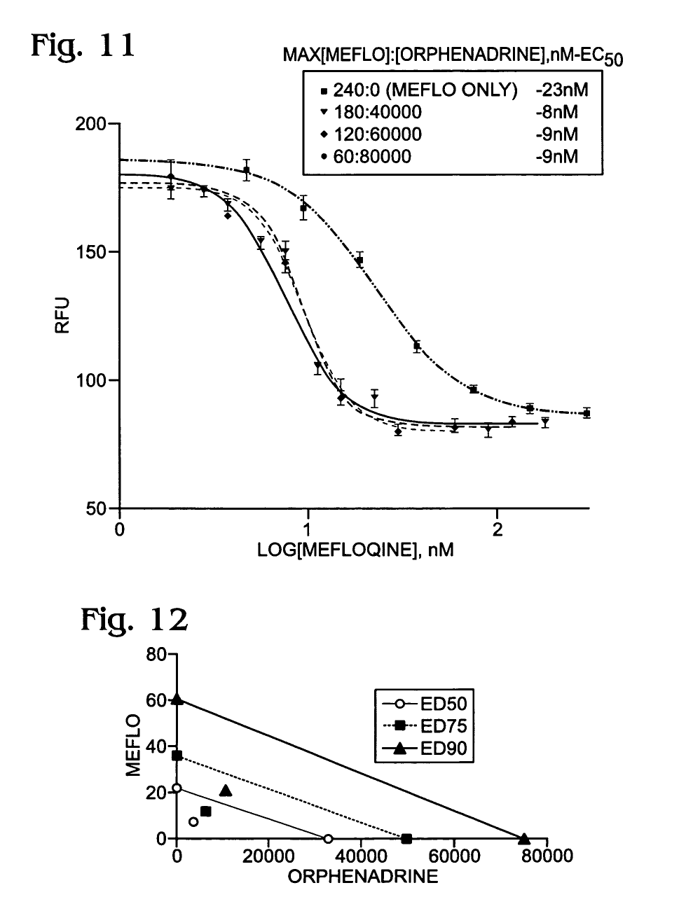

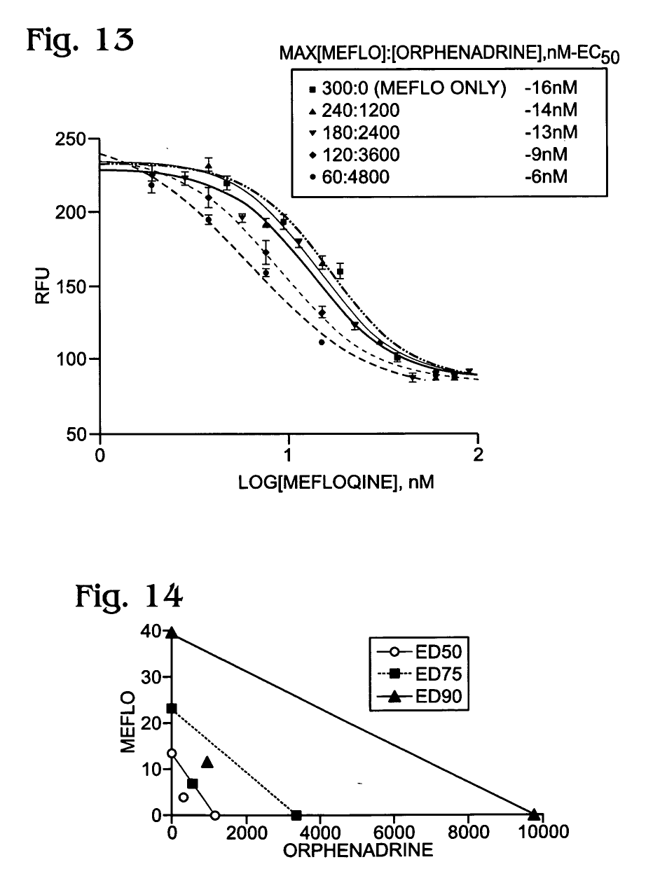

### DESCRIPTION AND OPERATION

Use of orphenadrine is proposed as part of combination therapy against drug-resistant malaria (specifically chloroquine-resistant P. falciparum), and also generally to other drug-resistance (e.g., quinine, halofantrine, mefloquine) and to other forms of malaria (e.g., P. vivax, P. ovale, P. malariae). In addition, functionally similar compounds are effective prophylaxis in rodent malaria models. It is also proposed to extend the application of orphenadrine as prophylactic treatment against malaria.

The invention includes use of the drug orphenadrine as a chemosensitizer or resistance-reversal agent to treat malaria or malignancy (whether using orphenadrine alone or in combination with other materials), and to provide prophylaxis against malaria. The invention includes treating malignancy based upon the observation of important similarities between multidrug-resistant cancer cells and drug-resistant malaria, both in mechanism and in the drugs that reverse the phenomenon. The same agents that reverse drug-resistance in malaria should show similar actions in some models of cancer drug-resistance. The invention includes these novel uses for orphenadrine, as well as any new formulations of orphenadrine in combination with antimalarial or antimalignancy drugs.

In addition, the invention includes structurally-similar orphenadrine analogues. In the case of other resistance-reversal agents with structures terminating in a dimethylamine or monomethylamine groups, the removal of one or both methyl groups preserves resistance-reversal function. For example there is no substantial difference in resistance-reversal potency between imipramine and desipramine, or between amitriptyline and nortriptyline. Therefore, it is also proposed that nororphenadrine (tofenacin) and dinororphenadrine will be active and thus included in this invention. In addition, because in vitro potency of diphenhydramine is similar to that of orphenadrine, the invention also proposes using analogues made by modifications at the phenyl rings of orphenadrine and their demethylated (nor-) and di-demethlyated amine forms.

### Representative Tests and Further Description of the Inventions

Since dose-related sedation precludes improving CP effect by simply increasing dose, we first sought an alternative by looking at brompheniramine (BP), pheniramine and fluorpheniramine (synthesized in our lab) in comparison with CP. All three halogenated pheniramines were roughly twice as potent as pheniramine, but pheniramine, by virtue of its poor antihistamine potency requires far higher dosing than BP or CP and achieves far higher concentrations (roughly 20-fold) in clinical use. Its superiority in achievable human concentration outweighs its inferiority in potency, so it was considered a candidate drug. Unfortunately, pheniramine shares sedation as a side effect, and possible superiority would be expected only to be incremental, in proportion to any dose (concentration) increase over CP.

In order to gain perhaps an additional log advantage over CP, we next investigated a stereospecific approach. Only the (+)enantiomers of the pheniramines are active as antihistamines but it is generally the racemic (+/−) form that is used clinically. We observed that the (+/−) and (+) forms of CP and BP were equipotent as C/RRs in vitro, indicating that the non-sedating (−)enantiomers would be active, and this was later confirmed using (−)CP. If, as suggested by known antihistamine toxicodynamics, toxicity other than sedation (e.g., cardiac conduction, other CNS effects) would not be expected unless much higher concentrations were reached, then the (−)enantiomers might be safe and tolerated at far higher doses than the (+/−) or (+) forms. If so, then even though no potency would be gained by a stereospecific approach, the much higher dosing made possible by the elimination of (+)enantiomer-mediated sedation would provide the desired log increase in effectiveness. This remains a viable concept, but requires an inexpensive production method for (−)CP, risks unexpected CNS or cardiovascular toxicity with CP dose escalation, and, as a new drug, would require extensive human testing.

The time and cost of taking on such a challenge demand that one be certain that the best candidate is receiving the investment. Because there are many drugs structurally and/or functionally related to the pheniramines that have a chiral carbon and thus can be separated into stereoisomers, we investigated their racemic forms to identify other candidates for the above approach. In addition, some non-chiral compounds were included for structure-activity relationships. Drugs tested include: antazoline, brompheniramine, carbinoxamine, cetirazine, chlorpheniramine, cyproheptadine, diphenhydramine, doxylamine, fluorpheniramine, homochlorcyclizine, hydroxyzine, meclizine, methapyrilene, orphenadrine, pheniramine, pyrilamine, and tripelennamine (these are in addition to drugs tested previously, mostly unrelated to the chiral concept: chlorthalidone, clomipramine, cyclobenzaprine, desipramine, doxepin, disopyramide, flumazenil, maprotiline, methazolamide, omeprazole, protriptyline, simetryn, trazodone, triamterene, verapamil, and several novel compounds synthesized in our lab).

Combining consideration of in vitro C/RR potency and known pharmacokinetics and dynamics, orphenadrine emerged as a superior candidate drug, expected to far outperform any previous C/RR agent or any of those tested by us. The in vitro data are presented below, but three further considerations must be kept in mind that amplify the importance of the results:

(1) Unlike desipramine and other tricyclic antidepressants (>90% protein bound) and CP (70% protein bound), orphenadrine is only 20% protein bound. Because in vitro testing is done in medium supplemented with only 10% serum by volume, the impact of protein binding is minimized, whereas in undiluted human serum the impact on free drug concentration is enormous.

(2) Typical serum orphenadrine concentrations in clinical use range from roughly 500 nM-2 μM (Labout et al, 1982), compared with <100 nM for CP, or 100-500 nM for desipramine.

(3) Orphenadrine does not typically cause sedation and achieves free drug concentrations in human use that are effective in vitro for C/RR. Therefore, the inexpensive, already available and extensively used racemic form is a viable candidate drug without any need for new drug development.

Combining the increased proportion of free drug and the increased achievable concentration, comparable or superior in vitro efficacy would be expected to translate into far superior clinical efficacy. Together with the in vitro results that follow, a conservative prediction would be that orphenadrine should be at least 10-100 times as effective as CP as a C/RR drug against malaria.

### Orphenadrine vs. Plasmodium—Test Results

All in vitro studies were 72 hour growth inhibition assays, starting with 0.2% parasitemia, 2% hematocrit under standard culture conditions in 96 well microplates. Growth was quantified using the fluorescence-based assay developed in our lab (Smilkstein et al; Antimicrobial Agents and Chemotherapy 2004;48:1803-1806). EC₅₀ determined by non-linear regression curve fitting (sigmoidal dose-response, variable slope equation) using GraphPad Prism software. Isobolograms and EC_{50,75 and 90} estimates computed using CalcuSyn software (Biosoft, T. C. Chou).

1. Like other C/RR drugs, intrinsic antimalarial activity of orphenadrine alone is far greater in multidrug resistant (e.g., Dd2) than in non-resistant (e.g., D6) parasites

2. Like other C/RR drugs, orphenadrine is synergistic with CQ against CQ-multidrug resistant parasites (e.g., W2, Dd2), but not against CQ sensitive D6.
a. In the following figures, fixed-ratio combinations of 2 drugs are made at the concentrations of each described in the figure legend (Fivelman, Adagu and Warhurst. Antimicrobial Agents and Chemotherapy 2004;48:4097-4102). Then, in quadruplicate, each of these combinations is diluted serially 2-fold, yielding one curve for each starting mixture. The data are expressed using the concentration of the antimalarial of interest on the X axis. The antimalarial EC50 values provided, therefore, are in combination with varying [orphenadrine] and are provided only to indicate the degree and direction of curve shift. In each starting combination, an increase of one drug is accompanied by a corresponding decrease in the other. As a result, overlapping curves indicate an additive relationship; a shift to the left, synergy; and a shift to the right, antagonism.
b. One representative isobologram from each of the experiments is included to the right of the figure, showing lines predictive of EC_{50,75 and 90} if the combinations were additive in effect. Points falling to the lower left of the predictive lines indicate synergy; those on or near the line, additive effects.

3. Orphenadrine is synergistic against parasites resistant to quinine (only Dd2 tested thus far) or mefloquine (D6 and Dd2 tested thus far). This is particularly intriguing because D6 lacks any of the mutations associated with PfCRT-mediated resistance, suggesting at least one non-PfCRT associated mechanism of action.

4. Orphenadrine is active in vivo against CQ-resistant P. chabaudi, but this effect is not yet adequately assessed. We have run a single in vivo trial in mice, as part of a dose-ranging pilot study. It was evident that orphenadrine alone decreased parasitemia at completion of 4 treatment days, and that mice treated with CQ and orphenadrine had lower parasitemia than those treated with CQ alone in the days following treatment completion. The small numbers of mice in each group, and the need to revise dosing preclude any further conclusions. We will be repeating the dose-ranging study, and subsequently will do a proper comparative trial.

5. Free drug concentrations comparable to predicted IC₉₀ values are achievable using orphenadrine. No other C/RR can approach this claim. The following table lists IC_{50,75 and 90} values calculated from among the fixed-ratio combination dose-response curves above, to provide some context for clinical consideration.

| | CQ/ | | |
|---|---|---|---|
| | orphenadrine | Quinine/orphenadrine | Mefloquine/orphenadrine |
| Dd2 | 17 nM/271 nM | 60 nM/721 nM | 4 nM/328 nM |
| IC₅₀ | | | |
| IC₇₅ | 26 nM/413 nM | 94 nM/1.1 μM | 7 nM/555 nM |
| IC₉₀ | 39 nM/631 nM | 146 nM/1.8 μM | 12 nM/938 nM |
| W2 | 31 nM/0.6 μM | — | — |
| IC₅₀ | | | |
| IC₇₅ | 50 nM/1 μM | — | — |
| IC₉₀ | 83 nM/1.7 μM | — | — |

For comparison, IC₉₀ values for CQ/(−)CP against W2 were 143 nM/2.9 μM. This CQ level is more difficult to achieve and the (−)CP concentration is 100-fold above achievable (+/−)CP concentration. In fact, to approach the free CP concentration present in the in vitro test would actually require a serum CP concentration roughly 300-fold above typical levels. In contrast, although typical values are <1 μM, documented steady-state orphenadrine concentrations in many patients on chronic therapy exceed any of the above, even when corrected for protein binding.

I claim:

1. A method of treating a malarial infection, comprising:
using orphenadrine as part of treating the infection.

2. The method of claim 1, wherein the using step involves using only orphenadrine.

3. The method of claim 1, wherein the using step involves using orphenadrine in combination with another antimalarial drug.

4. The method of claim 1, wherein the using step involves using orphenadrine in combination with plural antimalarial drugs.

5. The method of claim 1, wherein the using step involves using orphenadrine and chloroquine.

6. A method of providing prophylaxis against a malarial infection, comprising:
using orphenadrine as part of providing the prophylaxis.

7. The method of claim 6, wherein the using step involves using only orphenadrine.

8. The method of claim 6, wherein the using step involves using orphenadrine in combination with another antimalarial drug.

9. The method of claim 6, wherein the using step involves using orphenadrine in combination with plural antimalarial drugs.

10. The method of claim 6, wherein the using step involves using orphenadrine and chloroquine.

11. A method of treating a malignancy, comprising:
using orphenadrine as part of treating the malignancy.

12. The method of claim 11, wherein the using step involves using only orphenadrine.

13. The method of claim 11, wherein the using step involves using orphenadrine in combination with another antimalarial drug.

14. The method of claim 11, wherein the using step involves using orphenadrine in combination with plural antimalarial drugs.

15. The method of claim 11, wherein the using step involves using orphenadrine and chloroquine.
